(19) United States
(12) Patent Application Publication
(10) Pub. No.: US 2026/0104379 A1
(43) Pub. Date: Apr. 16, 2026

(54) PORTABLE SUSPENDED MICROORGANISM ANALYSIS DEVICE

(71) Applicant: KOREA INSTITUTE OF SCIENCE AND TECHNOLOGY, Seoul (KR)

(72) Inventors: Byoung Chan KIM, Seoul (KR); Cheulmin JOE, Seoul (KR); Sang Hyeon NOH, Seoul (KR)

(73) Assignee: KOREA INSTITUTE OF SCIENCE AND TECHNOLOGY, Seoul (KR)

(21) Appl. No.: 19/304,907

(22) Filed: Aug. 20, 2025

(30) Foreign Application Priority Data
Oct. 10, 2024 (KR) 10-2024-0137302

(51) Int. Cl. G01N 27/327; G01N 27/30; G01N 33/543

(52) U.S. Cl. CPC G01N 27/327; G01N 27/301; G01N 33/543; G01N 2469/10

(57) ABSTRACT

There is provided a portable suspended microorganism analysis device capable of integrating collection and analysis of microorganisms suspended in the atmosphere into a single apparatus, and thereby collecting and analyzing a target microorganism on site. The portable suspended microorganism analysis device, according to some embodiments, may include a collection unit configured to collect microorganisms suspended in the atmosphere, a connection part connected to the collection unit and configured to receive the microorganisms collected by the collection unit, an electrode unit connected to the connection part and configured to electrochemically analyze a reaction solution in which a solution accommodated in an internal space of the connection part is mixed with the microorganisms, to generate an electrical signal, and a portable analysis device configured to analyze and process the electrical signal from the electrode unit to generate data on a target microorganism.

### CROSS REFERENCE TO RELATED APPLICATION

The present application claims priority to Korean Patent Application No. 10-2024-0137302, filed on October 10 2024, in the Korean Intellectual Property Office, the entire contents of which are hereby incorporated by this reference.

### BACKGROUND OF THE INVENTION

### FIELD OF THE INVENTION

The present invention relates to a portable suspended microorganism analysis device, and more specifically, to a portable analysis device capable of collecting and analyzing microorganisms suspended in the atmosphere on site.

The present invention was conducted with the support of the following national research and development programs.

[National research and development program 1 that supported the present invention]

Project unique number: 2710034011

Project number: 2E33040

Name of department: Ministry of Science and ICT

Name of project management (specialized) institution: National Research Foundation of Korea

Name of research program: Korea Institute of Science and Technology Research Operating Expenses Support (Major Project Fund)

Name of research project: Atmospheric Environment Comprehensive Response Research Project

Name of project performing institution: Korea Institute of Science and Technology

Research period: January 1, 2024 to December 31, 2024

### DESCRIPTION OF THE RELATED ART

The conventional device and method for measuring microorganisms suspended in the atmosphere, generally collect air samples on site using a solid medium or a filter, and then transfer the collected samples to other equipment such as a laboratory, undergo a culturing process, and then analyze based on a colony count method or PCR.

FIG. 1 is a system for implementing a simulated state of suspended microorganisms in the atmosphere. With reference to FIG. 1, for example, a system 1000 provides a chamber system simulating an atmospheric environment. A system 1000 is configured to include an air bomb 1100 configured to introduce air in the atmosphere into the system at a predetermined pressure, a flow controller 1200 (mass flow controller, MFC) configured to control a flow rate of the air introduced from the air bomb 1100, a sprayer 1300 formed of a structure configured to spray a microbial solution by a high-speed air stream and to mix the microbial solution with the introduced air to spray in an aerosol form, a dryer 1400 formed of a moisture-absorbing structure including a desiccant to remove moisture by drying the sprayed air including microbial aerosol so as to simulate suspended microorganisms in the atmosphere, a filter 1500, and a chamber 1600 configured to simulate the suspended state in the atmosphere by distributing the air including microorganisms into a space having a predetermined volume.

By using the system 1000 configured to simulate a situation of atmospheric suspended microorganisms, it is impossible to detect, in real time on-site, the presence of microorganisms, since a separate process of analyzing by directly culturing on a medium through a sedimentation method, or analyzing after collecting suspended microorganisms in the atmosphere through an air sampler and separately culturing them on a solid medium, is required. That is, whether a target microorganism to be analyzed is appropriately collected may not be confirmed on site, and confirmation may only be possible through additional processes such as culturing, so the process is cumbersome, and considerable time is required. For example, in case of a colony count method, since a minimum culturing time of 24 hours or more is required, rapid detection of microorganisms and immediate response are not possible, and there is a disadvantage in that it is difficult to culture all microorganisms under various environmental conditions. In addition, for example, PCR-based analysis requires expensive equipment and professional personnel, so maintenance cost is relatively high, which is a disadvantage.

### SUMMARY OF THE INVENTION

One of the technical objects to be solved by the present invention is to provide an analysis device capable of efficiently collecting and analyzing microorganisms suspended in the atmosphere, regardless of location.

In addition, one of the technical objects to be solved by the present invention is to provide an analysis device capable of accurately detecting and analyzing the presence of a specific target microorganism.

The technical objects to be solved by the present invention are not limited to the objects described above, and other technical objects not mentioned will be clearly understood by those of ordinary skill in the art from the present specification and the accompanying drawings.

To achieve the aforementioned objects, there is provided a portable suspended microorganism analysis device, according to some embodiments of the present invention. The portable suspended microorganism analysis device may include a collection unit configured to collect microorganisms suspended in the atmosphere, a connection part connected to the collection unit and configured to receive the microorganisms collected by the collection unit, an electrode unit connected to the connection part and configured to electrochemically analyze a reaction solution in which a solution accommodated in an internal space of the connection part is mixed with the microorganisms, to generate an electrical signal, and a portable analysis device configured to analyze and process the electrical signal from the electrode unit to generate data on a target microorganism.

According to some embodiments, the electrode unit may include an electrode holder connected to an electrode terminal coupled to the portable analysis device, in which the electrode holder may include a connection hole formed to penetrate an upper surface and into which the connection part is inserted, and an electrode inserted into an electrode insertion hole formed to penetrate a side surface and electrically connected to the electrode terminal, and an internal space of the connection part inserted into the connection hole may be in fluid communication with the electrode.

According to some embodiments, the electrode holder may further include an O-ring hole, and an O-ring may be disposed in the O-ring hole to surround a side surface of an end of the connection part inserted into the connection hole.

According to some embodiments, the O-ring hole may include a first portion and a second portion, the first portion may be formed between the connection hole and the electrode insertion hole, the second portion may be formed to surround an outer side of the first portion, the O-ring may be disposed in the first portion, and the second portion may be spatially separated from the connection hole and the electrode insertion hole by the O-ring disposed in the first portion.

According to some embodiments, the electrode may include a working electrode having an aptamer fixed thereon to electrochemically react with a target microorganism included in the reaction solution, and in a state in which the electrode is inserted into the electrode insertion hole, the working electrode may be disposed at a position overlapping the internal space of the connection part in an up-down direction.

According to some embodiments, the electrode may further include a reference electrode configured to measure a potential change occurring at the working electrode and

a counter electrode configured to supply or collect current to complement an electrochemical reaction occurring at the working electrode, and the reference electrode and the counter electrode may be disposed at a position overlapping the second portion in the up-down direction.

According to some embodiments, the electrode holder may further include a clamp insertion hole formed below the electrode insertion hole, and a clamp may be inserted into the clamp insertion hole to press the electrode inserted into the electrode insertion hole and the O-ring.

According to some embodiments, the collection unit may include a collection part configured to be in fluid communication with the atmosphere to collect microorganisms suspended in the atmosphere and

a motor part based on a battery, configured to generate an air flow so that the microorganisms are introduced into the collection part.

According to some embodiments, the portable analysis device may be a portable potentiostat capable of visualizing the analyzed data.

According to some embodiments, the O-ring may be made of a material including rubber made of silicone or Aflas,

and a solution accommodated in the connection part may include phosphate-buffered saline.

Other detailed matters of the exemplary embodiment are included in the detailed description and the drawings.

According to embodiments of the present disclosure, microorganisms suspended in the atmosphere may be efficiently collected and analyzed on site.

In addition, according to embodiments of the present disclosure, the presence of a specific target microorganism may be accurately detected and analyzed.

In addition, according to embodiments of the present disclosure, evaporation of a solution mixed with microorganisms suspended in the atmosphere may be minimized, thereby enabling stable collection and analysis of the target microorganism.

In addition, according to embodiments of the present disclosure, simplification of the analysis device and minimization of maintenance cost for the analysis device may be achieved.

The effects of the present invention are not limited to the effects described above, and other effects, which are not mentioned above, will be clearly understood by those of ordinary skill in the art from the description of the claims.

### BRIEF DESCRIPTION OF THE DRAWINGS

FIG. 1 illustrates a system for implementing a simulated state of suspended microorganisms in the atmosphere.

FIG. 2 is a view schematically illustrating an analysis device according to an embodiment.

FIG. 3 is a perspective view schematically illustrating an electrode holder of FIG. 2.

FIG. 4 is a cross-sectional view schematically illustrating the electrode holder of FIG. 2.

FIG. 5 is a cross-sectional view schematically illustrating a state in which an electrode, an O-ring, and a clamp are coupled to the electrode holder of FIG. 2.

FIG. 6 is a plan view schematically illustrating the electrode of FIG. 5.

FIG. 7 is views schematically illustrating the clamp of FIG. 5.

FIG. 8 is a graph comparing the collection efficiency between a sedimentation method and a portable suspended microorganism analysis device according to an embodiment of FIG. 2.

FIG. 9 is a graph illustrating a change in electrochemical impedance according to a microbial concentration when using the portable suspended microorganism analysis device according to an embodiment of FIG. 2.

FIG. 10 is a graph illustrating a relationship between microbial concentration and a resistance change in an electrochemical reaction when using the portable suspended microorganism analysis device according to an embodiment of FIG. 2.

### DETAILED DESCRIPTION OF THE INVENTION

Hereinafter, embodiments of the present invention described below may be modified and implemented in various forms, and the technical spirit of the present invention is not limited to the embodiments described below. The terms used in the embodiments of the present invention have been selected, unless otherwise specifically defined in this specification by the applicant, by selecting general terms that are currently widely used while considering the functions in the invention. However, such terms may vary depending on the intention of a person skilled in the art to which the present invention pertains, court precedents, or the emergence of new technology. In addition, the terms or words used in the present specification and claims shall not be construed as being limited to their conventional or dictionary definitions, and shall be interpreted as including meanings and concepts that conform to the technical spirit of the present invention.

In the present specification, unless otherwise explicitly stated to the contrary, the expression that a certain component “includes” something shall be understood to imply not the exclusion of other components, but that it may further include other components. Specifically, terms "comprises," "comprising," "includes," "including," "containing," "has," "having" or other variations thereof shall be interpreted to denote the inclusion of features, numbers, steps, operations, components, parts, or combinations thereof as described in the present specification, and shall not be interpreted to preclude the possibility of the presence or addition of one or more other features, numbers, steps, operations, components, parts, or combinations thereof.

In the present specification, singular expressions shall be understood to include plural expressions unless clearly stated otherwise from the context. In addition, terms such as “first” and “second” may be used to describe various components, but the components shall not be limited by the terms, and the terms are only used to distinguish one component from another. A first component may be named as a second component within the scope belonging to the technical spirit of the present invention, and similarly, the second component may be named as the first component. In addition, in the drawings, shapes and sizes of components may be exaggerated to emphasize clear description. In addition, expressions such as “upper side,” “lower side,” “upper portion,” “lower portion,” “side surface,” “upper surface,” and “lower surface” described below are based on the directions shown in the drawings, and it is pre-disclosed that such expressions may vary when the orientation of the corresponding object is changed. Further, in the present specification, the terms “module,” “part,” or “unit” may denote a unit composed of a single element, or a unit expressed as a combination or set of a plurality of elements.

Hereinafter, embodiments of the present invention will be described in detail with reference to the drawings attached so that a person having ordinary skill in the technical field to which the present invention pertains may easily carry out the present invention.

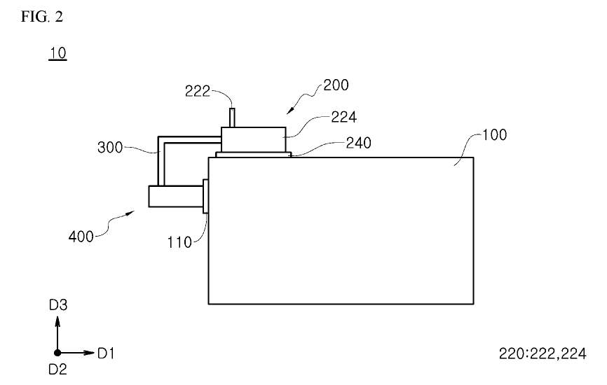

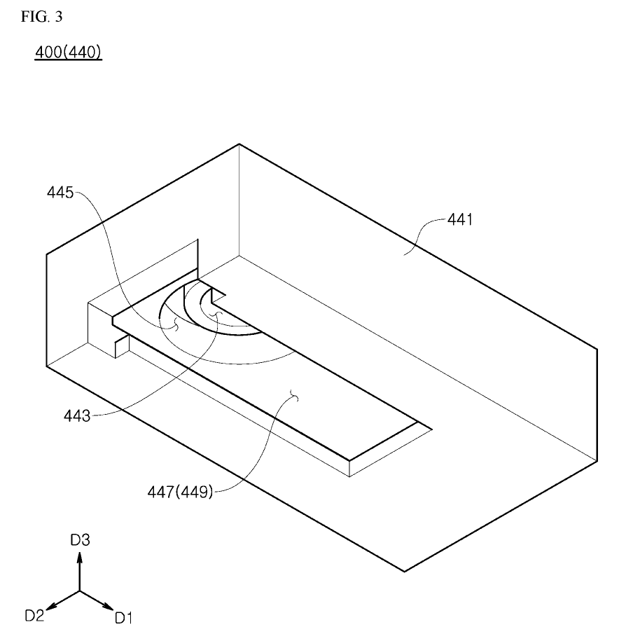

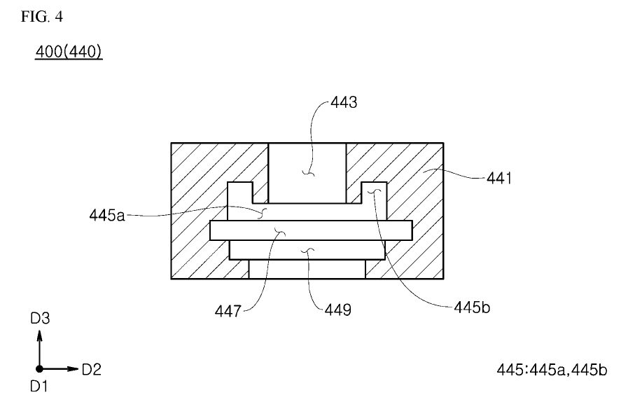

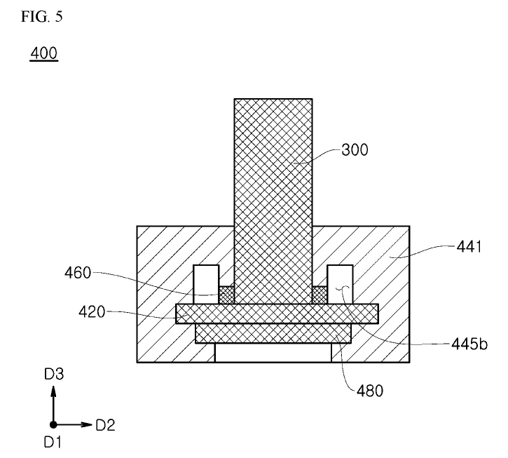

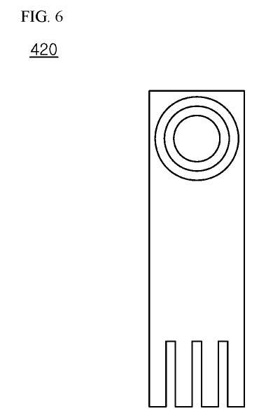

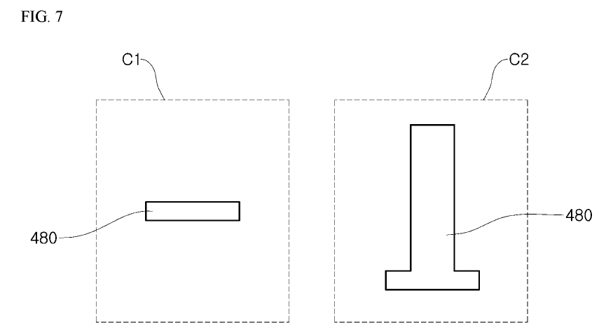

FIG. 2 is a view schematically illustrating an analysis device according to an embodiment. FIG. 3 is a perspective view schematically illustrating an electrode holder of FIG. 2. FIG. 4 is a cross-sectional view schematically illustrating the electrode holder of FIG. 2. FIG. 5 is a cross-sectional view schematically illustrating a state in which an electrode, an O-ring, and a clamp are coupled to the electrode holder of FIG. 2. FIG. 6 is a plan view schematically illustrating the electrode of FIG. 5. FIG. 7 is views schematically illustrating the clamp of FIG. 5. In FIG. 7, C1 illustrates a front view of a clamp according to some embodiments, and C2 illustrates a top view of the clamp.

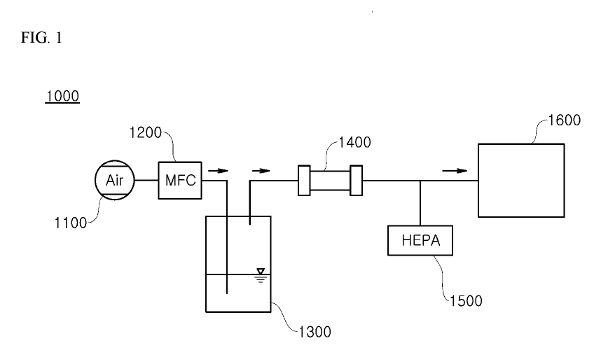

With reference to FIGS. 2 to 7, a portable suspended microorganism analysis device 10 according to some embodiments may collect microorganisms suspended in the atmosphere, and may analyze the collected microorganisms. In addition, the portable suspended microorganism analysis device 10 according to some embodiments may detect the suspended microorganisms in a chamber 1600 (see FIG. 1) by using the system 1000 (see FIG. 1) that simulates the atmospheric suspended microorganism situation described above. According to some embodiments, the portable suspended microorganism analysis device 10 may have a size and weight such that a user may carry it. Accordingly, the portable suspended microorganism analysis device 10 may not only collect microorganisms suspended in the atmosphere directly on site, but also may analyze the collected microorganisms on site. That is, the portable suspended microorganism analysis device 10 may integrally perform both collection and analysis of microorganisms at a single location. For example, the portable suspended microorganism analysis device 10 may diagnose and analyze microorganisms electrochemically by using a bio-receptor. Specifically, the portable suspended microorganism analysis device 10 may be configured to detect microorganisms suspended in the atmosphere on site by integrating a method of sampling from the atmosphere and a method of detecting the sampled specimen. In addition, the portable suspended microorganism analysis device 10 may detect and analyze a specific target microorganism to be collected and analyzed. Detailed description thereof will be provided below.

The portable suspended microorganism analysis device 10 according to some embodiments may include a portable analysis device 100, a collection unit 200, a connection part 300, and an electrode unit 400. In some embodiments, the portable analysis device 100 and the electrode unit 400 may be arranged in one direction. Hereinafter, for ease of understanding, the direction in which the portable analysis device 100 and the electrode unit 400 are arranged is defined as a first direction D1, and a direction perpendicular to the first direction D1 when viewed from above is defined as a second direction D2. In addition, a direction perpendicular to a plane including both the first direction D1 and the second direction D2 is defined as a third direction D3. For example, the third direction D3 may refer to a direction perpendicular to the ground.

The portable analysis device 100 according to some embodiments may be a portable device configured to analyze and process data on microorganisms collected and analyzed by the collection unit 200 and the electrode unit 400 to be described below, and may be capable of visualizing such data. For example, the portable analysis device 100 may be a portable potentiostat. In some embodiments, the portable analysis device 100 may be provided based on a battery, and may be provided as a portable device operable independently without external power supply, and may be linked with various mobile devices such as smartphones, tablets, and laptops through wireless functions using Bluetooth, Wi-Fi, etc. or wired functions using a USB connection terminal, etc. Specifically, the portable analysis device 100 may be linked to a mobile device and may analyze microorganisms in real time by transmitting data to the mobile device or the like, and may display analysis result data such as graphs and charts on the mobile device.

The collection unit 200 according to some embodiments may collect microorganisms suspended in the atmosphere. In some embodiments, the collection unit 200 may include a collector 220 and a fixing plate 240.

The collector 220 according to some embodiments may be fixed to the portable analysis device 100. Specifically, the collector 220 may be fixed to the portable analysis device 100 through the fixing plate 240 coupled to the portable analysis device 100. For example, the fixing plate 240 may be coupled to a ceiling wall of the portable analysis device 100 by a fixing means (for example, a screw, etc.), but the technical spirit of the present invention is not limited to such examples. For example, the collector 220 may be coupled to the portable analysis device 100 by a fixing means other than screws.

The collector 220 according to some embodiments may include a collection part 222 and a motor part 224. The collection part 222 according to some embodiments may be configured to be exposed to the atmosphere. For example, one end of the collection part 222 may be coupled to an upper end of the motor part 224 to be configured to be in communication with the motor part 224, and the other end may be formed to be exposed to the atmosphere. For example, the collection part 222 may have a structure extending along the third direction D3 from one end to the other end. In some embodiments, the collection part 222 may have a generally cylindrical shape in which a hollow is formed therein. Accordingly, microorganisms suspended in the atmosphere may be collected through the collection part 222, and the collected microorganisms may be introduced into the motor part 224.

The motor part 224 according to some embodiments may generate a flow of air at a predetermined speed so that microorganisms in the atmosphere are introduced into the collection part 222. For example, the motor part 224 may be a motor-based air flow sampler. In some embodiments, according to the flow of air generated by the motor part 224, microorganisms in the atmosphere may be introduced into the collection part 222, and the microorganisms introduced into the collection part 222 may be delivered to the connection part 300, which will be described below, through the motor part 224.

The motor part 224 according to some embodiments may be configured to include a motor, a power supply device, an Arduino, and a motor driver. In some embodiments, the motor of the motor part 224 may be a DC motor. For example, the motor of the motor part 224 may be a DC motor of 4.5 V and 500 mA, capable of generating an air flow of up to 2.5 LPM(Liters Per Minute), but is not limited thereto. The power supply device according to some embodiments may be a rechargeable and dischargeable battery. For example, the power supply device of the motor part 224 may have a structure in which a plurality of batteries are connected in series to supply a voltage of 6 V. In addition, the Arduino according to some embodiments may generate a pulse width modulation (PWM) signal to control the speed of the motor of the motor part 224. The generated PWM signal may control the cycle of an electrical signal, and may adjust the rotation speed (RPM) of the motor of the motor part 224, thereby controlling the strength of the air flow introduced into the motor part 224. In addition, in some embodiments, the motor driver may receive a PWM signal generated by the Arduino, and may control the rotation speed of the motor of the motor part 224 based on the signal. That is, by adjusting the rotation speed of the motor of the motor part 224 according to various field conditions (for example, wind strength, etc.), microorganisms suspended in the atmosphere may be efficiently collected, and appropriate power may be supplied so that the motor of the motor part 224 is not overloaded or overheated, thereby enabling stable control of the operation of the motor part 224.

In some embodiments, the collection part 222 may be formed at a location adjacent to the connection part 300, which will be described below. For example, when viewed from the third direction D3, the collection part 222 may be positioned adjacent to the connection part 300. Accordingly, air introduced into the collection part 222 may be delivered to the connection part 300 through the motor part 224.

The connection part 300 according to some embodiments may be connected to the collector 220 and the electrode unit 400, respectively. Specifically, one end of the connection part 300 may be connected to the motor part 224, and the other end may be connected to a connection hole 443 formed in an electrode holder 440, which will be described below. In some embodiments, the connection part 300 may have a shape that is generally a right-angle bend (‘ㄱ’ shape). In addition, a cross-sectional shape of the connection part 300 may be generally circular. However, the present invention is not limited thereto, and the shape of the connection part 300 may be modified into various shapes that may connect the collector 220 and the electrode unit 400 to each other within the scope of the technical spirit of the present invention. In addition, a space through which air may flow may be formed inside the connection part 300. The space formed inside the connection part 300 may be in fluid communication with the collection part 222 through the motor part 224.

In some embodiments, a solution may be filled in the internal space of the connection part 300. According to some exemplary embodiments, the solution may be filled in a partial space of the internal space of the connection part 300. For example, the solution may be filled only in the internal space of the connection part 300 that extends along the third direction D3, and the solution may be delivered to the electrode unit 400, which will be described below, and may not be delivered to the motor part 224. Accordingly, while damage to the motor, the battery, etc. constituting the motor part 224 may be prevented by the solution, the air flow including microorganisms suspended in the atmosphere may come into contact with and be mixed with the solution filled in the connection part 300. According to some embodiments, the solution filled in the internal space of the connection part 300 may be a buffer solution. Specifically, the solution filled in the internal space of the connection part 300 may be a buffer solution configured to maintain osmotic pressure, pH, and/or ion concentration constant. For example, such a solution may be a phosphate-buffered saline (1xpbs) solution. However, the present invention is not limited thereto, and the type of solution filled in the internal space of the connection part 300 may be variously modified depending on the type of target microorganism to be collected and analyzed.

The electrode unit 400 according to some embodiments may generate an electrical signal by electrochemically reacting with a reaction solution in which microorganisms and a solution are mixed. The electrical signal generated by the electrode unit 400 may be transmitted to the portable analysis device 100 described above. The portable analysis device 100 may receive the electrical signal transmitted from the electrode unit 400, and may analyze and process the signal to generate data on the microorganisms.

The electrode unit 400 according to some embodiments may include an electrode 420, an electrode holder 440, an O-ring 460, and a clamp 480.

The electrode 420 according to some embodiments may generate an electrical signal. Specifically, the electrode 420, as described above, may generate an electrical signal by electrochemically reacting with a reaction solution in which microorganisms and a solution are mixed. According to some exemplary embodiments, the electrode 420 may be a 3-cell electrode in which an aptamer, composed of single-stranded DNA and/or RNA capable of selectively binding (recognizing) a specific target microorganism, is immobilized. Specifically, the electrode 420 may be a 3-cell electrode system composed of a working electrode, a reference electrode, and a counter electrode. In some embodiments, the working electrode of the electrode 420 may include gold (Au), the reference electrode may include silver/silver chloride (Ag/AgCl), and the counter electrode may include platinum (Pt).

For example, a gold working electrode according to some embodiments may be an electrode that causes an electrochemical reaction with a target microorganism, and may be an electrode where an electrical signal is generated. Specifically, the aptamer may be stably maintained in a state fixed to a surface of the working electrode through a gold-thiol bond, and accordingly, the aptamer may interact with a target microorganism with high selectivity through binding, thereby changing the characteristics of the electrode surface. In addition, the reference electrode according to some embodiments may be an electrode configured to stably maintain a reference potential during an electrochemical reaction. That is, the reference electrode may provide a reference point of potential change of the electrode by measuring a potential change that occurs at the working electrode during the electrochemical reaction. In addition, the counter electrode according to some embodiments may be an electrode configured to supply or collect current in order to complement the electrochemical reaction occurring at the working electrode. The counter electrode may receive or provide current on the opposite side of the working electrode to complete the overall circuit, thereby facilitating the electrochemical reaction of the target microorganism.

The electrode holder 440 according to some embodiments may fix the electrode 420. In some embodiments, the electrode holder 440 may include a body 441, a connection hole 443, an O-ring hole 445, an electrode insertion hole 447, and a clamp insertion hole 449.

The body 441 according to some embodiments may have a generally cuboidal shape. In addition, the body 441 may be connected to the portable analysis device 100. For example, the body 441 may be coupled to an electrode terminal 110 installed on one of sidewalls of the portable analysis device 100. In this case, a longitudinal direction of the body 441 may be directed generally toward the first direction D1, and the electrode insertion hole 447, which will be described below, and the electrode terminal 110 may be positioned to be overlapped with each other in the first direction D1. Accordingly, as will be described in detail below, the electrode 420 inserted into the electrode insertion hole 447 may be fitted into the electrode terminal 110, and the electrode 420 may be electrically connected to the portable analysis device 100 through the electrode terminal 110 to transmit and receive an electrical signal.

The body 441 according to some embodiments may have a plurality of holes formed therein. According to some exemplary embodiments, the plurality of holes formed in the body 441 may include a connection hole 443, an O-ring hole 445, an electrode insertion hole 447, and a clamp insertion hole 449.

In some embodiments, the connection hole 443, the O-ring hole 445, the electrode insertion hole 447, and the clamp insertion hole 449 may be sequentially positioned in the third direction D3. For example, the connection hole 443, the electrode insertion hole 447, and the clamp insertion hole 449 may be sequentially positioned in a direction from an upper side to a lower side in the third direction D3.

In the connection hole 443 according to some embodiments, the connection part 300 described above may be inserted. The connection hole 443 may penetrate an upper end of the body 441. In addition, when viewed from the third direction D3, the connection hole 443 may be formed at a position spaced apart from a central portion of the body 441. However, the present invention is not limited thereto, and the position at which the connection hole 443 is formed on the body 441 may be variously modified to be a position at which the connection part 300 may be structurally and stably inserted into the connection hole 443. A cross sectional shape of the connection hole 443 according to some embodiments may be generally circular. In addition, a diameter of the connection hole 443 may correspond to a diameter of the connection part 300 described above. Accordingly, the other end of the connection part 300 may be inserted into the connection hole 443.

In addition, the O-ring hole 445 according to some embodiments may be configured to include a first portion 445a and a second portion 445b. The first portion 445a and the second portion 445b may be formed integrally. In addition, the first portion 445a may be positioned between the connection hole 443 and the electrode insertion hole 447 in the third direction D3. A cross sectional shape of the first portion 445a may be generally circular. The second portion 445b according to some embodiments may be formed to surround an outer side of the first portion 445a. The second portion 445b may have a generally ring shape. In addition, an upper end of the second portion 445b may be positioned above the upper end of the first portion 445a in the third direction D3. In addition, a lower end of the second portion 445b may be positioned at a height corresponding to a lower end of the first portion 445a in the third direction D3. As will be described below, the O-ring 460 may be disposed in the first portion 445a, and the second portion 445b may be provided as a buffer space. That is, the second portion 445b may be spatially separated from the holes 443, 447, and 449 by the O-ring 460 disposed in the first portion 445a. Detailed description thereof will be provided below.

The electrode 420 described above may be inserted into the electrode insertion hole 447 according to some embodiments. In addition, the electrode insertion hole 447 may be formed at a position overlapping with the connection hole 443 and the O-ring hole 445 in the third direction D3. In addition, the electrode insertion hole 447 may generally have a shape corresponding to that of the electrode 420. For example, the electrode insertion hole 447 may have a generally rectangular shape. In addition, in some embodiments, the electrode insertion hole 447 may be positioned between the O-ring hole 445 described above and the clamp insertion hole 449, which will be described below. According to some exemplary embodiments, the electrode insertion hole 447 may be formed to penetrate both one side surface of the body 441 and the other side surface thereof facing the one surface. For example, the electrode insertion hole 447 may be formed to have a longitudinal direction parallel to the first direction D1. In a state in which the electrode 420 is inserted into the electrode insertion hole 447, a working electrode of the electrode 420 may be positioned to overlap the connection hole 443 in the third direction D3.

A clamp 480, which will be described below, may be inserted into the clamp insertion hole 449 according to some embodiments. As described above, the clamp insertion hole 449 may be positioned below the electrode insertion hole 447 in the third direction D3. The clamp insertion hole 449 may have a shape generally corresponding to that of the clamp 480. In addition, the clamp insertion hole 449 may be formed to penetrate one side surface of the body 441, but may be formed not to penetrate the other side surface facing the one side surface. Similar to the electrode insertion hole 447, the clamp insertion hole 449 may be formed at a position overlapping the connection hole 443 in the third direction D3.

The O-ring 460 according to some embodiments may include a material having excellent chemical resistance. In some embodiments, the O-ring 460 may include a material that does not react with a reaction solution accommodated in the connection part 300. For example, a material of the O-ring 460 may include rubber made of silicone or Aflas. In addition, the O-ring 460 may be inserted into the O-ring hole 445. Specifically, in some embodiments, the O-ring 460 may have a generally ring shape. In addition, the O-ring 460 according to some embodiments may be disposed in the first portion 445a. For example, as illustrated in FIG. 5, in a state in which the connection part 300 is inserted into the connection hole 443 described above, the O-ring 460 may be disposed to surround a side surface of the other end (an end) of the connection part 300. That is, by forming the O-ring hole 445, particularly the first portion 445a, in which the O-ring 460 is disposed between the connection hole 443 and the electrode insertion hole 447, a pressing force of the connection part 300 and the body 441 with respect to the O-ring 460 may be enhanced. Accordingly, the reaction solution accommodated in the connection part 300 may be airtightly sealed such that it is in contact only with the electrode 420 inserted into the electrode insertion hole 447, so that leakage of the reaction solution may be prevented and evaporation of the reaction solution may be minimized, thereby making it easier to collect microorganisms. In addition, when simply dropping a reaction solution onto the electrode 420, only a small volume of the solution may be supplied onto the electrode 420, but according to the above-described some embodiments, the connection part 300 may be airtightly sealed by the O-ring 460, so the volume of the reaction solution supplied to the electrode 420 may be increased.

In addition, the second portion 445b according to some embodiments may be separated from other holes 443, 447, and 449 by the O-ring 460 disposed in the first portion 445a, as described above. Accordingly, the second portion 445b may function as a buffer space that may minimize direct contact between a reference electrode and a counter electrode of the electrode 420 and the electrode holder 440 when the electrode 420 is inserted into the electrode insertion hole 447. That is, in a state in which the electrode 420 is inserted into the electrode insertion hole 447, the reference electrode and the counter electrode of the electrode 420 may be disposed at a position overlapping the second portion 445b in the third direction D3. Accordingly, accurate measurement of a target microorganism included in the reaction solution may be enabled by the electrode 420.

The clamp 480 according to some embodiments may have a generally rectangular shape so as to efficiently press the electrode 420 inserted into the electrode insertion hole 447. In addition, an end of the clamp 480 may have a protruding shape toward a side thereof. For example, the clamp 480 may generally have a ‘T’-shape. In some embodiments, when the clamp 480 is inserted into the clamp insertion hole 449, the end of the clamp 480 may come into contact with a side surface of the body 441 and may be fixed thereto.

As illustrated in FIG. 5, the connection part 300 may be inserted into the connection hole 443, the electrode 420 may be inserted into the electrode insertion hole 447, and the clamp 480 may be inserted into the clamp insertion hole 449. In this case, after the electrode 420 is first inserted into the electrode insertion hole 447, the clamp 480 may be inserted into the clamp insertion hole 449. In this process, airtightness between the connection part 300 and the electrode 420 may be secured by the O-ring 460 disposed in the first portion 445a, and a working electrode of the electrode 420, which is disposed to overlap in the third direction D3, may be positioned to be in fluid communication with the connection part 300. Accordingly, the reaction solution accommodated in the internal space of the connection part 300 may be easily delivered to the working electrode of the electrode 420. In addition, due to the second portion 445b spatially separated by the O-ring 460, the reference electrode and the counter electrode of the electrode 420 may minimize contact with the reaction solution and the body 441, so that microorganisms may react intensively with the working electrode. In addition, since the electrode 420 may be more firmly fixed and pressed by the clamp 480 inserted into the clamp insertion hole 449, an airtight effect by the O-ring 460 described above may be further enhanced.

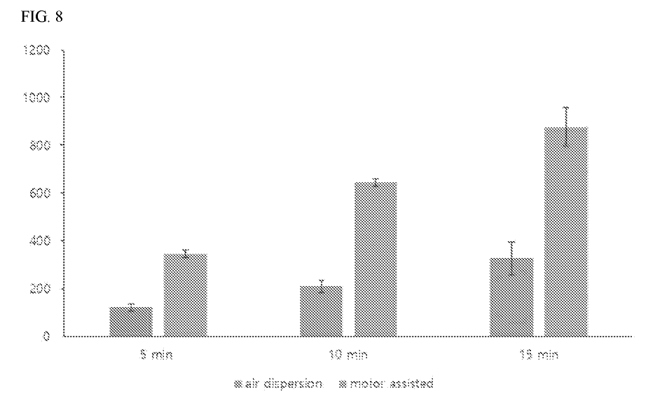

FIG. 8 is a graph illustrating a comparison between a sedimentation method using the system of FIG. 1 and a collection efficiency of the analysis device according to the embodiment of FIG. 2. Here, a horizontal axis may represent collection time, and a vertical axis may represent a collection amount of a target microorganism. In addition, a blue graph may represent a case in which microorganisms are collected using the sedimentation method under the condition of FIG. 1, and an orange graph may represent a case in which microorganisms are collected using the portable suspended microorganism analysis device 10 according to some embodiments of FIG. 2.

With reference to FIG. 8, when microorganisms are collected using the portable suspended microorganism analysis device 10 (see FIG. 2, etc.) according to some embodiments, it can be confirmed that collection efficiency is improved by approximately three times compared to the case using the sedimentation method. That is, the portable suspended microorganism analysis device 10 according to some embodiments may form an air flow at a predetermined speed in the motor part 224 to efficiently collect microorganisms suspended in the atmosphere, and may immediately guide the collected microorganisms to the electrode holder 440 for real-time analysis.

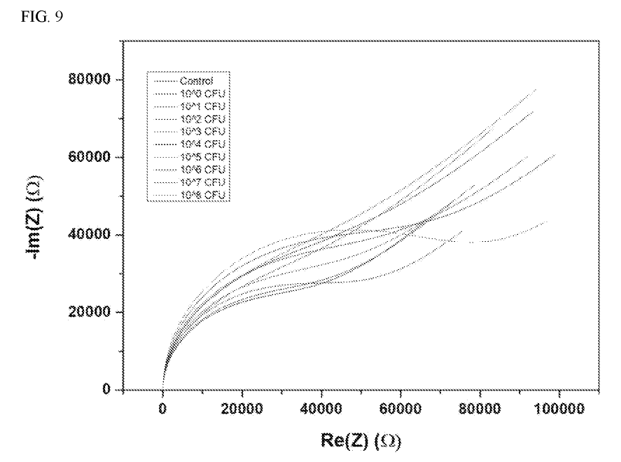

FIG. 9 is a graph illustrating a change in electrochemical impedance according to a microbial concentration when using the portable suspended microorganism analysis device according to an embodiment of FIG. 2. The graph of FIG. 9 is a Nyquist plot, where the horizontal axis may represent a real component (Re Z, resistance component), and the vertical axis may represent an imaginary component (-Im Z, inductive or capacitive component).

As illustrated in FIG. 9, it can be seen that, as the microbial concentration increases (in the order of 10⁶, 10⁷, 10⁸ CFU, etc.), the curve becomes more bent and shows an upward pattern, compared to a control under a condition without microorganisms. That is, as the microbial concentration increases, microorganisms in the reaction solution may be largely fixed and react on the aptamer on the surface of the working electrode of the electrode 420, so the electrochemical resistance increases, and accordingly, a tendency of increased capacitance is shown. As described above, the portable suspended microorganism analysis device 10 according to some embodiments may allow the connection part 300, in which a reaction solution is accommodated, to be tightly in contact with the electrode 420 by the O-ring 460 and the clamp 480, so that the amount and/or concentration of the reaction solution delivered to the electrode 420 may be improved. Accordingly, electrochemical resistance increases in response to the increase in microbial concentration, and a signal-to-noise ratio (SNR) transmitted to the portable analysis device 100 may be improved, thereby improving the accuracy of target microorganism analysis in the portable analysis device 100.

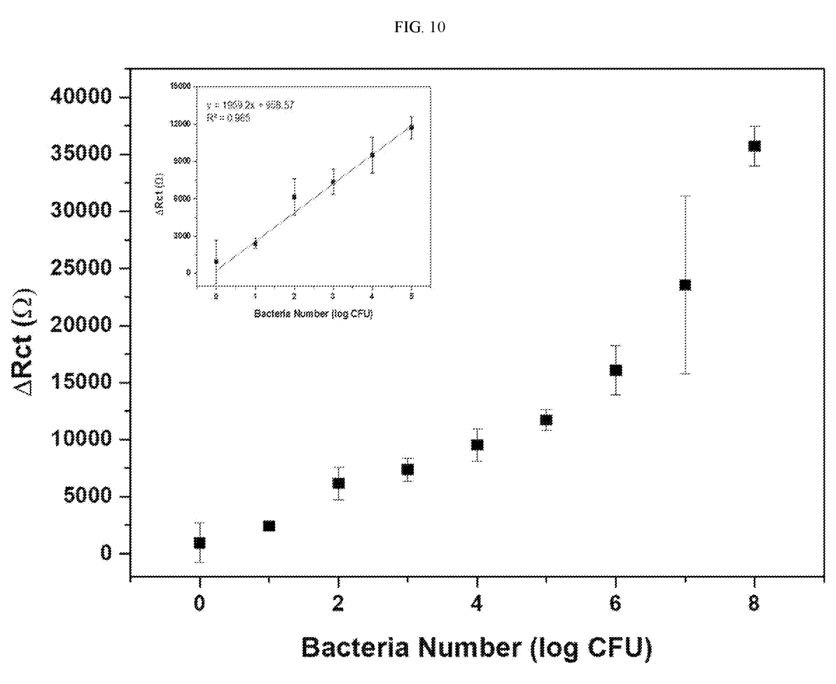

FIG. 10 is a graph illustrating a relationship between microbial concentration and a resistance change in an electrochemical reaction when using the portable suspended microorganism analysis device according to an embodiment of FIG. 2. A horizontal axis of FIG. 10 represents microbial concentration (log scale CFU), and a vertical axis may represent an amount of change in resistance.

With reference to FIG. 10, low-concentration microorganisms (log CFU 0 to 4) show relatively low values of the amount of change in resistance, and high-concentration microorganisms (log CFU 5 to 8) show a tendency of rapid increase in values of the amount of change in resistance. Such a tendency particularly shows that as the microbial concentration increases in the high concentration region, resistance in the electrochemical reaction greatly increases. That is, by improvement in tightness between the connection part 300 and the electrode 420 due to the O-ring 460 and the clamp 480 according to the above-described some embodiments, the clarity of an electrical signal generated in the electrode unit 400 may be improved, thereby enhancing the accuracy of target microorganism analysis in the portable analysis device 100.

The foregoing detailed description illustrates the present disclosure. Further, the foregoing description merely shows and describes the exemplary embodiments of the present disclosure, and the present disclosure can be used in various other combinations, modifications, and environments. That is, alterations or modifications may be made within the scope of the concept of the invention disclosed in the present specification, the scope equivalent to the described disclosure, and/or the scope of the technology or knowledge in the art. The disclosed embodiments are provided to explain the best state for implementing the technical spirit the present disclosure, and various modifications required for the specific fields of application and the use of the present disclosure may be made. Thus, the detailed description of the present disclosure is not intended to limit the present disclosure to the disclosed embodiments. Moreover, the appended claims should be construed to include other embodiments.

### Description of Reference Numerals

10: Portable suspended microorganism analysis device

100: Portable analysis device 200: Collection unit

220: Collector 224: Motor part

300: Connection part 400: Electrode unit

420: Electrode 440: Electrode holder

460: O-ring 480: Clamp

What is claimed is:

1. A portable suspended microorganism analysis device, comprising:
a collection unit configured to collect microorganisms suspended in the atmosphere; a connection part connected to the collection unit and configured to receive the microorganisms collected by the collection unit;
an electrode unit connected to the connection part and configured to electrochemically analyze a reaction solution in which a solution accommodated in an internal space of the connection part is mixed with the microorganisms, to generate an electrical signal; and
a portable analysis device configured to analyze and process the electrical signal from the electrode unit to generate data on a target microorganism.

2. The portable suspended microorganism analysis device of claim 1, wherein the electrode unit includes an electrode holder connected to an electrode terminal coupled to the portable analysis device,
wherein the electrode holder includes:
a connection hole formed to penetrate an upper surface and into which the connection part is inserted; and
an electrode inserted into an electrode insertion hole formed to penetrate a side surface and electrically connected to the electrode terminal, and
wherein the internal space of the connection part inserted into the connection hole is in fluid communication with the electrode.

3. The portable suspended microorganism analysis device of claim 2, wherein the electrode holder further includes an O-ring hole, and
wherein an O-ring is disposed in the O-ring hole to surround a side surface of an end of the connection part inserted into the connection hole.

4. The portable suspended microorganism analysis device of claim 3, wherein the O-ring hole includes a first portion and a second portion,
the first portion is formed between the connection hole and the electrode insertion hole,
the second portion is formed to surround an outer side of the first portion,
the O-ring is disposed in the first portion, and
the second portion is spatially separated from the connection hole and the electrode insertion hole by the O-ring disposed in the first portion.

5. The portable suspended microorganism analysis device of claim 4, wherein the electrode includes a working electrode having an aptamer fixed thereon to electrochemically react with a target microorganism included in the reaction solution, and
wherein in a state in which the electrode is inserted into the electrode insertion hole, the working electrode is disposed at a position overlapping the internal space of the connection part in an up-down direction.

6. The portable suspended microorganism analysis device of claim 5, wherein the electrode further includes:
a reference electrode configured to measure a potential change occurring at the working electrode; and
a counter electrode configured to supply or collect current to complement an electrochemical reaction occurring at the working electrode,
and
wherein the reference electrode and the counter electrode are disposed at a position overlapping the second portion in the up-down direction.

7. The portable suspended microorganism analysis device of claim 3, wherein the electrode holder further includes a clamp insertion hole formed below the electrode insertion hole, and
a clamp is inserted into the clamp insertion hole to press the electrode inserted into the electrode insertion hole and the O-ring.

8. The portable suspended microorganism analysis device of claim 1, wherein the collection unit includes:
a collection part configured to be in fluid communication with the atmosphere to collect microorganisms suspended in the atmosphere; and
a motor part based on a battery, configured to generate an air flow so that the microorganisms are introduced into the collection part.

9. The portable suspended microorganism analysis device of claim 1, wherein the portable analysis device is a portable potentiostat capable of visualizing the analyzed data.

10. The portable suspended microorganism analysis device of claim 4, wherein the O-ring is made of a material including rubber made of silicone or Aflas, and
a solution accommodated in the connection part includes phosphate-buffered saline.
